(19) United States
(12) Patent Application Publication
(10) Pub. No.: US 2007/0190582 A1
(43) Pub. Date: Aug. 16, 2007

(54) Platelet surface PF4 antigenic complexes as a diagnostic indicator in heparin-induced thrombocytopenia

(76) Inventors: Mortimer Poncz, Philadelpia, PA (US); Douglas B. Cines, Wynnewood, PA (US); Gowthami Arepally, Durham, NC (US); Lubica Rauova, Philadelpia, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/613,787

(22) Filed: Dec. 20, 2006

Related U.S. Application Data
(60) Provisional application No. 60/751,822, filed on Dec. 20, 2005.

(51) Int. Cl. G01N 33/567

(52) U.S. Cl. 435/7.21

(57) ABSTRACT

A method for diagnosing a predisposition for developing HIT is provided.

This invention claims priority under 35 U.S.C. §119(e) to U.S. Provisional Application 60/751,822 filed Dec. 20, 2005, the entire contents being incorporated by reference herein as though set forth in full.

Pursuant to 35 U.S.C. §202(c) it is acknowledged that the U.S. Government has certain rights in the invention described, which was made in part with funds from the National Institutes of Health, Grant Numbers HL54500, HL054749 and HL69471.

### FIELD OF THE INVENTION

This invention relates to the field of medicine. More specifically, the invention provides methods for assessing a patient's risk for developing heparin-induced thrombocytopenia (HIT).

### BACKGROUND OF THE INVENTION

Several publications and patent documents are cited throughout the specification in order to describe the state of the art to which this invention pertains. Each of these citations is incorporated herein by reference as though set forth in full.

Heparin-induced thrombocytopenia (HIT) is an iatrogenic complication of heparin therapy caused by antibodies that recognize complexes formed between heparin and Platelet Factor 4 (PF4) (1-3). The thrombocytopenia that develops is often mild, but approximately half of the patients develop life- or limb-threatening thrombosis (4-6). Management involves careful monitoring of platelet counts, a high index of clinical suspicion, cessation of heparin exposure, and the introduction of alternative anticoagulants based on a compatible clinical presentation (7,8). These measures have reduced the incidence of new thromboembolic complications, but have had less impact on the incidence of amputations and death (9,10). Heparin remains an important anticoagulant in widespread use, and studies that help define the pathophysiology of HIT may lead to better identification of patients at risk and more targeted treatment strategies that may be more effective rather than reliance on systemic anticoagulants.

The antibody response in HIT is unusual in several respects. First, the major complications of HIT are related to thrombosis, in clear contrast to other drug-induced thrombocytopenias (11). This high incidence of thrombosis may be related to the ability of HIT antibodies to activate platelets via FcγRIIA in addition to their capacity to activate endothelial cells and monocytes (12,13). In a murine model of HIT, only mice that expressed both human (h) PF4 and FcγRIIA on their platelets developed thrombocytopenia and thrombosis when given an anti-heparin:PF4 HIT-like monoclonal antibody (mAb) KKO (14). A second unusual feature is the surprisingly high incidence of anti-PF4/heparin antibodies in heparinized patients, approaching a quarter to over half of all exposed patients in some settings (15-18). Why only a small portion of these patients develop HIT is not clear and no unequivocal differences in the specifics of the antibody response between the vast majority of individuals who remain asymptomatic and the small number who develop HIT have been identified, although differences in IgG titers have been noted (19).

A third unusual feature of HIT antibodies (including KKO) is that they bind optimally to heparin:PF4 complexes over a very narrow molar ratio in vitro (1-3,20). In the case of unfractionated high molecular weight (HMW) heparin, PF4 forms ultralarge complexes (ULC) of >670 kDa at these same molar ratios (21). These ULC are stable, are particularly antigenic, bind multiple IgG antibodies/complex and promote platelet activation by KKO (21).

### SUMMARY OF THE INVENTION

In accordance with the present invention, a method for diagnosing a patient's predisposition for the development of heparin-induced thrombocytopenia (HIT) is provided. An exemplary method entails obtaining a cell sample from a patient and determining the level of total platelet factor 4 (PF4) and HIT antigen expression on the surface of said cells, elevated levels of PF4 being indicative of a predisposition for the development of HIT. Such cells include without limitation, endothelial cells, circulating monocytes, and platelets. In a preferred embodiment, total PF4 levels, e.g., surface and internal stores (e.g., PF4 stored in alpha granules) in platelets is determined. A preferred embodiment of the method includes the use of anti-PF4 antibody to assess total surface PF4 . The method may optionally comprise treating the patient sample with PGE1 to inhibit spontaneous activation and release of PF4 from the platelets. The method optionally comprises determining IgG antibody titers in the patient. In yet another aspect of the method of the invention, the platelet surface is assessed for the presence of antigenic complexes comprised of PF4 and cell surface glycosaminoglycans. Thus, the invention provides methods for determining total and antigenic cell surface PF4 levels.

In yet another aspect of the invention, a method is provided for identifying agents having efficacy for the treatment of HIT. An exemplary method entails providing cells which express GAG-PF4 antigenic complexes on the surface, incubating the cells in the presence and absence of the agent, and identifying those agents which disrupt GAG-PF4 antigenic complexes, said agents having efficacy for the treatment of heparin induced thrombocytopenia, with the proviso that said agent is not heparin or protamine sulfate.

### BRIEF DESCRIPTION OF THE DRAWINGS

FIG. 1. Binding of mAb to human platelets in the presence of added hPF4 . (A) The graphs show the fold-increase in the mean fluorescence intensity (MFI) of antibody binding in the presence and absence of the noted concentrations of PF4 . Open diamonds: TRA isoimmune control. Grey squares: antihuman CD41 mab. Black circles: KKO. KKO is a HIT-like monoclonal antibody that enables us to study the biologic features relevant to HIT antibodies in a controlled fashion. Each antibody was added at 50 μg/mL. (B) The fold-change in antigenicity for KKO in the presence of PF4 at 12.5 μg/mL (open diamonds), 50 μg/mL (grey squares) and 200 μg/mL (black circles), with heparin added at the concentrations shown. The Y-axis indicates fold change from that at baseline without heparin. (C) Platelet activation by KKO (50 μg/mL) at the indicated PF4 concentrations as measured by Annexin V binding. (D) Kinetics of KKO binding (50 μg/mL) in the presence of 50 μg/mL of PF4 . The mean +1 standard deviation (SD) is shown for the experiments performed three times, each in triplicate.

FIG. 2. KKO binding to murine platelets in the presence of hPF4 . (A) The same as FIG. 1A, but mPF4 null platelets were studied with increasing amounts of added hPF4 . Open diamonds: total platelet PF4 . Grey squares: total surface immunogenic PF4 . Black circles: KKO-detectable surface PF4 . (B) Same as in (A) for KKO, but using mPF4 null platelets pre-treated with CS ABC. (C) Studies as in (A) for KKO, but genotype of the mice are as shown and the platelets were incubated with either the FcγRIIA blocking mAb IV.3 or an isotype control (50 μg/mL) for 30 min at RT prior to addition of PF4 and KKO. (D) Studies are as in (A) for KKO with the genotype of the mice indicated. Relative annexin binding was measured. (E) Time course of KKO and RTO binding (50 μg/mL) in the whole blood samples to transgenic murine platelets. +/+=hPF4High/FcγRIIA+double transgenic mouse platelets. +/−=hPF4 High/FcγRIIA-transgenic mouse platelets. MFI is in absolute values. For (A)-(D), the mean ±1 SD is shown. Each experiments was performed three times, each in triplicate. In (E), a study representative of three is shown.

FIG. 3. Platelet activation by HIT IgG. (A) Squares represent IgG isolated from 4 HIT plasmas incubated with human platelets that had been exposed to different amounts of hPF4 . Black circles=simultaneously studied KKO and open circles=simultaneously studied isoimmune control TRA. (B) Same as (A) using a commercial control IgG preparation (open diamonds) or 4 preparations of IgG from normal controls (black and grey-shaded diamonds). The mean value is shown for each patient studied on 3-5 separate occasions, each experiment done in duplicate. For clarity, standard deviations are not shown.

FIG. 4. Characterization of hPF4 mice. (A) Total platelet-associated hPF4 expressed per mL of blood in WT animals and the three hPF4 transgenic mice lines studied. Controls (Ctl) were platelets from 4 human donors. The mean ±1 SD is shown for the experiments performed three times, each in triplicate. (B) Flow cytometric measurement of CD41+-platelet-bound FITC-KKO in the same animals as in (A) measured 10 min after IV-injected FITC-KKO.

FIG. 5. KKO-induced thrombocytopenia in hPF4 mice. (A) Platelet counts in mice after IP injection of KKO. First time point is at 3 hr post injection. Black circle hPF4 High mice, 200 μg KKO; and white circle=FcγRIIA transgenic mice, 200 μg KKO. Diamonds=hPF4 High/FcγRIIA double transgenic mice. White to light gray to dark gray to black diamonds=50, 100, 200 and 400 μg KKO IP, respectively. The mean of 3 experiments, each performed in triplicate, is shown. (B) Animals were all hPF4 High/FcγRIIA double transgenic mice. Open circle=200 μg TRA, IP; black circles=200 μg RTO, IP; and black diamond=200 μg KKO, IP. The mean ±1 SD of 3 experiments, each in triplicate is shown. (C) All animals received 200 μg KKO. Black and white circles as in (A). Open diamond=hPF4 Low; grey diamond=hPF4 Mid; and black diamond=hPF4 High. The mean ±1 SD of 3 experiments, each in triplicate is shown. *=p<0.05 from baseline value. (D) All animals received 200 μg KKO, IP. Black circle as in (A). Grey diamond=hPF4 Mid mice and black diamond=hPF4 Mid mice that also received 20 U heparin SQ daily for 4 days as indicated by arrows. The mean ±1 SD of 3 experiments, each in triplicate is shown. *=p<0.05 of heparin treated from untreated hPF4 Mid mice.

FIG. 6 Therapeutic intervention in HIT model. KKO (200 μg) was given IP at baseline preceded by either IV heparin (100 U/kg) or protamine sulfate (2 mg/kg). Platelets counts were measured at the times noted. Subsequent therapeutic interventions at 21 and 45 hrs are denoted by vertical gray arrows. (A) hPF4 Mid/FcγRIIA animals. (B) hPF4 High/FcγRIIA animals. KKO only animals=gray diamonds. KKO plus heparin=open triangles. KKO plus protamine sulfate=black circles. At least 4 animals were studied per time point. The means ±1 SD are shown. *=p, 0.05 vs. animals receiving KKO alone.

FIG. 7. Schematic representation of the induction of HIT model. The situation shown at the top is more common. Patients have low or normal levels of total platelet PF4 and if they have atherosclerosis or other causes of vascular injury leading to platelet activation and PF4 release, they have relatively low levels of surface PF4 expression. When these patients are heparinized, PF4 is removed, fewer antigenic complexes remain, and there is less likelihood of platelet activation if HIT antibodies develop. These patients are at low risk of HIT. The bottom shows the smaller subset of patients with high levels of total PF4 who have suffered significant vascular injury and/or significant platelet activation and have high surface PF4 levels. Upon heparinization, they form and retain significant amounts of antigenic complexes on the platelet surface and if they develop HIT antibodies, they are at high risk of developing HIT.

### DETAILED DESCRIPTION OF THE INVENTION

Heparin-induced thrombocytopenia (HIT) antibodies recognize complexes between heparin and Platelet Factor 4 (PF4 ). Heparin and PF4 bind HIT antibodies only over a narrow molar ratio. The involvement of platelet surface-bound PF4 as an antigen in the pathogenesis of experimental HIT has been examined. In accordance with the present invention, we show that HIT antibodies also recognize complexes between PF4 and cell surface glycosaminoglycans and that such cell surface PF4 complexes are also antigenic only over a restricted concentration range of PF4 . Heparin is not required for HIT antibody binding, but shifts the concentration of PF4 needed for optimal surface antigenicity to higher levels. These data are supported by in vitro studies involving both human and murine platelets with exogenous recombinant human (h) PF4 , and either an anti-PF4 /heparin monoclonal antibody KKO or HIT immunoglobulin. Injection of KKO into transgenic mice expressing different levels of hPF4 demonstrates a correlation between platelet hPF4 and HIT antigen expression and the severity of the thrombocytopenia. Therapeutic interventions in this model using high-dose heparin or protamine sulfate support the pathogenic role of surface PF4 antigenic complexes in the etiology of HIT. We believe that this focus on surface PF4 advances our understanding of the pathogenesis of HIT and provides means to identify patients at high risk to develop HIT upon heparin exposure.

### DEFINITIONS

“Heparin-induced Thrombocytopenia” (HIT) refers to a transient, heparin-induced autoimmune disorder which occurs as a complication of therapy in 1-5% of patients treated with unfractionated high molecular weight (HMW) heparin.

“Platelet factor 4”, (PF4 ) is a member of the CXC subfamily of chemokines that possesses high affinity for heparin and other large anionic molecules. In accordance with the present invention surface expression levels exceeding 50 μg/mL are associated with a risk of developing HIT.

“Hit antigen” refers to an antigenic complex of PF-4 and glycosaminoglycans.

The following example is provided to illustrate certain embodiments of the invention. It is not intended to limit the invention in any way.

The materials and methods set forth below are provided to facilitate the practice of the present invention.

Preparation of Recombinant WT hPF4 .

Wildtype (WT) human (h) PF4 in pT7-7 plasmid was expressed in BL21DE30 pLysS bacteria, purified, and characterized as described (25). Recombinant protein was isolated from bacterial lysate supernatant by affinity chromatography using a HiTrap Heparin HP column (Amersham Bioscience). Proteins were purified further by FPLC using a RESOURCE®RPC column (Amersham Bioscience). Protein purity was assessed by 15% (wt/vol) sodium dodecyl sulfate (SDS)-polyacrylamide gel electrophoresis (PAGE) followed by silver staining (26). Samples were subjected to immunoblotting after electrotransfer to polyvinylidenedifluoride (PVDF) membranes using rabbit anti-hPF4 polyclonal antibody (PeproTech), followed by donkey anti-rabbit secondary antibody conjugated to horseradish peroxidase (HRP) (Jackson ImmunoResearch Laboratories) and developed using the ECL kit (PerkinElmer Life Sciences). Total protein concentrations were determined using the bicinchoninic acid assay (Pierce) as per manufacturer with BSA as standard.

Monoclonal Antibodies and HIT Immunoglobulins.

KKO, the anti-hPF4 specific mAb RTO and isoimmune control TRA are all mouse IgG2b mabs (27). Antibodies were fluorescein isothiocyanate (FITC) labeled using a E-Z FITC labeling kit (Pierce) as per the manufacturer. Monoclonal anti-human CD41a PerCP-Cy5 and anti-mouse CD-41 phycoerythrin (PE) antibodies and Annexin V-PE were from Pharmingen. Polyclonal IgG was isolated from the plasma of 4 patients with clinical HIT (7,8,11,28) and a positive HIT enzyme-linked immunosorbent assay (ELISA) (3), and from 4 healthy subjects with recombinant protein G-Agarose (Invitrogen), as per the manufacturer. A commercial human IgG preparation (Pierce) was used as an additional control. HIT IgG reactivity with heparin:PF4 complexes was confirmed by ELISA (3).

Platelet Preparation and Analyses.

Studies were performed using human platelets or platelets from WT and transgenic mice (see below for the description of the mice). Human blood was collected after informed consent from healthy, aspirin-free volunteers in acid citrate dextrose (ACD, pH 4.5, 10:1, vol/vol) under a protocol approved by the Institutional Review Board for Studies involving Human Subjects of the Children's Hospital of Philadelphia. All studies involving mice were approved by the same Institution's Institute Animal Care and Use Committee. Blood was centrifuged at 200 g for 15 min at room temperature (RT) to generate platelet-rich plasma (PRP). PGE₁ (final concentration 1 μg/mL, Sigma) was added to the PRP to prevent spontaneous platelet activation. PRP was centrifuged at 800 g for 10 min at RT, and the pellet washed and resuspended in modified Tyrode's buffer (134 mM NaCl, 3 mM KCl, 0.3 mM NaH₂PO₄, 2 mM MgCl₂, 5 mM HEPES, 5 mM glucose, 12 mM NaHCO₃, 0.1% BSA (Sigma A7030, fatty acid-free)). Mouse PRP and washed platelets were prepared using blood collected from the inferior vena cava in ACD (1:5 vol:vol), immediately diluted 1:3 (vol:vol) in modified Tyrode's buffer containing PGE1 (final concentration, 1 μg/mL) and centrifuged at 200 g for 4 min at RT. PRP was centrifuged at 800 g for 10 min at RT, and the pellet washed and resuspended in modified Tyrode's buffer. Both human and murine washed platelets were used at 10⁸/mL. Washed platelets were incubated with varying amounts (0-80 μg) of recombinant human (h) PF4 in a final volume of 100 μL for 45 min at RT. In some experiments, increasing amounts of unfractionated heparin (0-40 μg, porcine intestinal mucosa, Sigma) was added to the platelets before the PF4 . KKO or another antibody (50 μg) under study was then added to each sample for an additional 15 min. Samples were then diluted with Tyrode's buffer and enumerated immediately or fixed in 1% paraformaldehyde in PBS (1:10 vol/vol). Total immunodetectable platelet hPF4 was determined using murine platelets completely deficient in murine PF4 (mPF4 null (29)). PF4 was crosslinked on the platelet surface by adding 1% paraformaldehyde (1:10 vol/vol) overnight at 4° C. Samples were washed with PBS, and platelets were lysed in NuPage LDS Sample Buffer (Invitrogen). Fractions were separated on a 10% SDS-PAGE gel and immunoblotted after electrotransfer to a PVDF membrane with a rabbit antihuman PF4 (1:5,000) primary antibody (PeproTech) followed by an HRPconjugated donkey anti-rabbit antibody (Jackson ImmunoResearch Laboratories) and developed using an ECL kit. Autoradiograpic bands over the linear range of exposure were analyzed on a UMAX Vista-58 scanner, and the data analyzed by developing a histogram of the calculated density using the NIH program ImageJ (rsb.info.nih.gov). Total surface PF4 binding was also detected using mPF4 null platelets after paraformaldehyde crosslinking, as above. Samples were then washed 3× with Tyrode's buffer and incubated with polyclonal rabbit anti-hPF4 antibody, and followed by FITC conjugated goat anti-rabbit IgG. Binding of KKO was performed by indirect immunofluorescence as a control. Samples were incubated with unlabeled KKO after a 1hr incubation with PF4 , then fixed with 1% paraformaldehyde overnight at 4° C., washed 3× with Tyrode's buffer and stained with FITC conjugated goat anti-mouse IgG. Washed mouse platelets (PF4 KO mice) were incubated with various concentrations (0-5 U/ml) of chondroitinase ABC (Sigma) and/or heparinase I (Sigma) at 37° C. After 30 min, aliquots containing equal numbers of platelets was incubated with Tyrode's buffer containing various concentrations of PF4 (0-400 μg/mL, final concentration) for 60 mm at RT. FITC-labeled KKO (50 μg/mL) was added for 15 min, the sample was diluted 1/10 with Tyrode's buffer and antibody binding was measured by flow cytometry as above.

Platelet Flow Cytometry.

Binding of FITC-labeled KKO to the platelet was identified using a Becton Dickinson FACSscan calibrated for fluorescence and light scatter using the manufacturer's standard beads (CaliBRITE, Becton Dickinson). Data for forward-angle scatter (FSC), side-angle scatter (SSC) and fluorescence were obtained with gain settings in logarithmic mode. Human platelets were identified and gated according to the SSC and immunofluorescence with anti-CD41a mAb. Platelet activation was estimated by both Annexin V binding and P-selectin expression (23). To measure the binding of Annexin V, the incubated platelets were diluted 1:10 in binding buffer (0.01M Hepes, 0.14M NaCl, and 2.5 mM CaCl₂) containing Annexin V-PE. When Annexin V-PE and KKO-FITC binding were measured simultaneously, platelets were size-selected based on side- and forward-scatter.

Characterization of Transgenic Mice.

Transgenic mice expressing different amounts of hPF4 mRNA per platelet have been described previously (26). Three lines bearing 1, 6 and 22 copy numbers of the human PF4 gene/haplotype were used. Previous analysis of multiple tissues using immunohistochemistry and RT-PCR showed that hPF4 was expressed exclusively in megakaryocytes. Transgenic mice expressing FcγRIIA were generously provided by Steven McKenzie, Thomas Jefferson University (30) and crossbred with these hPF4 mice. All murine lines were backcrossed onto the C57BL/6J background>8 times. Genomic makeup of mice was determined by PCR analysis using oligonucleotide primers described previously (26,30). Controls included littermates transgenic for hPF4 or FcγRIIA only. Mice were 6-10 weeks of age at the time of study. Total platelet hPF4 levels in the various transgenic hPF4 lines were determined using an Asserachrom PF4 kit (Diagnostica Stago) as per the manufacturer using recombinant hPF4 as the standard. Mouse blood was obtained by retroorbital puncture. The plate was read at 450 nm in a THERMOmax microplate reader (Molecular Devices). Measurements of surface KKO binding in vivo in WT and the hPF4 transgenic mice were determined after IV injection of 20 μg of FITC-labeled KKO in 200 μL of sterile PBS via the tail vein followed by withdrawal of 50 μL of blood from the retroorbital plexus 10 min later. The blood was coimmunostained for CD41, and KKO binding to CD41-positive cells was estimated by flow cytometry. In other studies, KKO was injected intraperitoneally (IP) in a final volume of 200 μL diluted with sterile PBS. Porcine heparin (200 μL of 100 U/mL stock; Abbott Laboratories) was injected subcutaneously (SQ) in a subgroup of studied animals beginning at 24 hrs for 4 consecutive days. Complete blood counts were measured in 50 μl of whole blood obtained by retroorbital puncture into Safe-T-Fill® minicapillary blood collection tubes (Kabe Labortechnik). Platelets were enumerated using an automatic cell counter (HEMAVET, Drew Scientific). In the therapeutic intervention studies, either porcine heparin (100 U/kg) or protamine sulfate (2 mg/kg) were injected IV over 2 mins. KKO (200 μg) was given IP 1 hr later (zero time point). Injection of heparin or protamine was repeated 21 and 45 hr later. Blood counts were determined as above.

Statistics.

Platelet counts between groups were compared using the Student's t-test. Statistical analyses were performed using Graph Pad Prism (Graphpad Software). Differences were considered significant at a p value of <0.05.

### Results

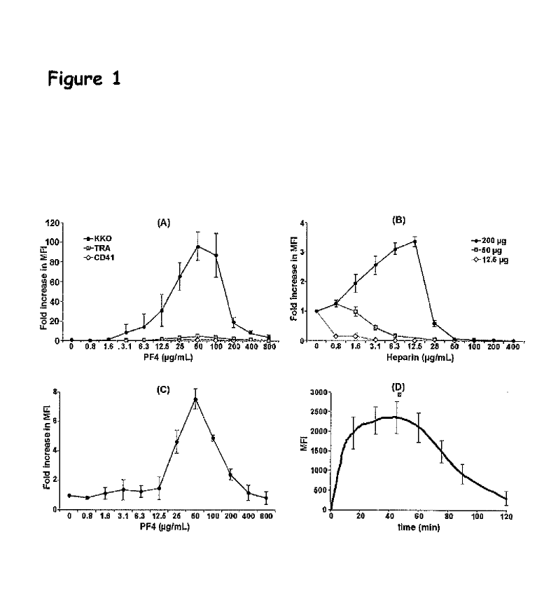

PF4 bound to the platelet surface forms antigenic complexes on human platelets. To better understand the pathogenesis of HIT, we asked whether antigenic complexes form between PF4 and GAGs on the platelet surface. KKO bound poorly to unstimulated, washed human platelets (FIG. 1A). However, addition of recombinant hPF4 markedly increased binding of KKO in a dose-dependent manner. Binding followed a bell-shaped curve (FIG. 1A). Maximal binding of KKO, corresponding to an ˜100-fold increase in fluorescence intensity, occurred at an hPF4 concentration of 50 μg/mL. This peak was not limited by the amount of KKO added (data not shown). Binding of an isotype control mAb TRA and anti-CD41 mAb increased <7% compared with KKO over the same range of PF4 concentrations (FIG. 1A).

We next examined the effect of heparin on the binding of KKO to surface-bound PF4 . Platelet GAGs are composed predominantly of chondroitin and, to a lesser extent, heparan sulfates (31), each of which has a lower affinity for PF4 than HMW heparin (32). At levels of added PF4 where binding of KKO to platelets is suboptimal (left side of the curve in FIG. 1A), binding was reduced further or eliminated by addition of heparin. FIG. 1B shows this result at a low level of surface hPF4 (12.5 μg/mL added, open diamonds, FIG. 1B) and for the peak level of surface hPF4 (50 μg/mL added, grey squares, FIG. 1B). However, in the presence of hPF4 concentrations that exceeded peak antigen formation on platelets, addition of heparin enhanced KKO binding. FIG. 1B shows this for 200 μg/mL hPF4 (black circles, FIG. 1B). These studies suggest that in settings associated with high levels of surface-bound PF4 , heparin enhances cell surface antigenicity.

Binding of KKO to PF4 -coated platelets induced their activation as measured both by an increase in surface binding of Annexin V (FIG. 1C) and expression of P-selectin (data not shown). We then asked whether activation releases additional PF4 from internal stores, which in turns alters the composition of GAG:PF4 complexes and KKO binding. To examine this possibility, we incubated human platelets with 50 μg/mL of hPF4 and followed KKO binding over time. KKO binding increased with time, reaching a plateau at 20 to 60 min and then decreased (FIG. 1D). These dynamic changes suggest that the composition of the surface GAG:PF4 complexes had been modified over time, possibly due to release of PF4 from newly recruited FcγRIIA-activated platelets (14). As additional PF4 is incorporated into these complexes, the optimal ratio is exceeded and antibody binding is impaired.

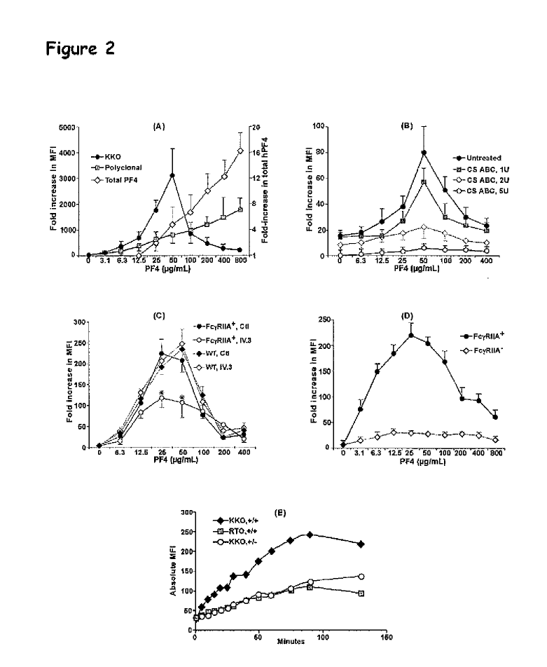

PF4 bound to the platelet surface forms antigenic complexes on murine platelets. Studies of human platelets are thus confounded by the release of internal stores of hPF4 and the presence of FcγRIIA on their surface. We, therefore, switched to murine platelets that naturally lack the FcγRIIA platelet receptor, and studied KKO binding to surface of mPF4 null platelets (29). Addition of hPF4 lead to a near-doubling in total platelet PF4 for each doubling of hPF4 in the media over the range studied (FIG. 2A, open diamonds) with a slightly blunted, but similar, proportional increase in total surface immunogenic PF4 detected using a polyclonal anti-hPF4 antibody (FIG. 2A, grey squares). Under similar conditions, binding of KKO to murine platelets followed the same bell-shaped curve seen with human platelets (FIG. 2A, black circles).

We then examined whether KKO recognized PF4 bound to surface GAGs by pretreating the cells with either chondroitinase (CS) ABC or heparinase 1 or both together. CS ABC alone (FIG. 2B) or with heparinase 1 (data not shown), but not heparinase 1 alone (data not shown), decreased KKO binding. These data are consistent with platelet membrane GAGs being composed predominantly of chondroitin sulfates (31,32). When GAGs were stripped from the platelet surface, the concentration of PF4 needed for maximal KKO binding was unaltered. This may indicate that clusters of chondroitin remain intact, while other areas of the platelet become devoid of GAGs.

We then determined whether FcγRIIA contributed to the binding of KKO. Murine platelets from WT animals or FcγRIIA⁺ transgenic animals were incubated with increasing concentrations of hPF4 . The amount of KKO bound in the presence of IV.3, an FcγRIIA blocking antibody or an isotype control (12) was then measured. Binding of KKO to WT and FcγRIIA⁺ platelets followed the same bellshaped curve in the presence of the isotype control, consistent with binding through the Fab end of the molecule (FIG. 2C). However, IV.3 did reduce the total amount of KKO that bound to FcγRIIA⁺ platelets only (FIG. 2C), suggesting the presence of the FcγRIIA receptor may also provide stability to bound KKO.

Platelet activation by KKO clearly leads to platelet activation via FcγRIIA engagement, as WT murine platelets are minimally activated, as measured by the binding of Annexin V (FIG. 2D). We then studied the effect of platelet activation on KKO binding using murine platelets that were double transgenic for high levels of hPF4 and FcγRIIA, hPF4^{High}/FcγRIIA⁺ (+/+in FIG. 2E), compared to hPF4^{HIGH}/FcγRIIA⁻ platelets (+/−in FIG. 2E). hPF4^{High}/FcγRIIA⁺ and hPF4^{High}/FcγRIIA⁻0 platelets bound the same amount of KKO at time zero. However when FcγRIIA was expressed, binding of KKO increased greatly over time (FIG. 2E). A much smaller increase was also seen for the binding of RTO, a mAb that binds to hPF4 independent of heparin (27) (FIG. 2E). These studies support the concept that platelet activation via FcγRIIA releases additional PF4 that becomes incorporated within antigenic complexes recognized by KKO.

Studies with HIT IgG.

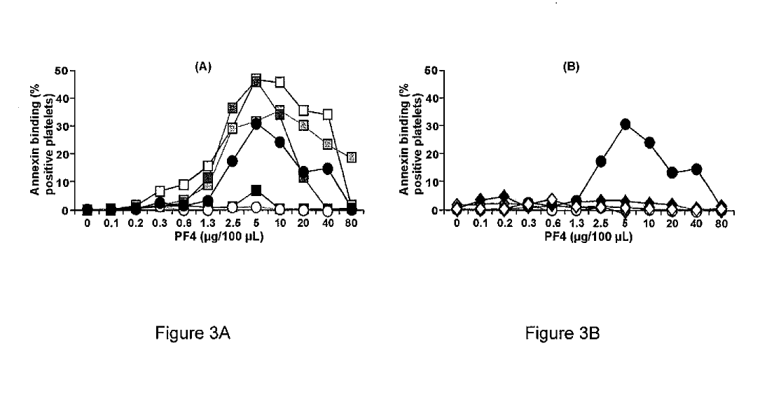

KKO competes with many HIT antibodies for binding to platelets, suggesting a common epitope on heparin:PF4 , and activates platelets through similar mechanisms (27). Nevertheless, we extended our studies to determine whether HIT antibodies behaved in a similar manner with respect to platelet surface PF4 levels. Studies based on those shown in FIG. 1A were repeated using either IgG isolated from patients with HIT diagnosed by clinical criteria (7,8,11,28) and a positive HIT ELISA (3), IgG from normal volunteers, or a commercial pooled IgG preparation. Three of the 4 HIT IgG samples tested caused strong activation of platelets as measured by binding of Annexin V (FIG. 3A) in contrast to the four normal controls or a commercial IgG preparation (FIG. 3B). Maximal platelet activation occurred at the same concentration of PF4 (50 μg/mL) as was seen with KKO.

In vivo Studies in Mice Expressing Different Amounts of hPF4 .

The in vitro data indicate that there is an amount of cell surface PF4 at which HIT-antibody binding is maximal. This bell-shaped relationship between PF4 concentration and binding of HIT antibody extends previous studies in which a similar relationship was seen when the concentrations of PF4 and heparin in solution were varied (22). However, GAGs appear to fulfill the role of heparin on the platelet surface. We proposes that in a murine model of HIT: 1) the severity of thrombocytopenia should parallel endogenous hPF4 expression, 2) if sufficient PF4 has already been released and bound to the cell surface, exogenous heparin would not be required to cause thrombocytopenia once antibody is present, and 3) heparin would exacerbate thrombocytopenia in the setting of high PF4 content.

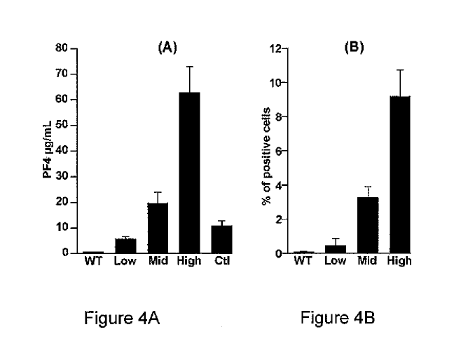

We previously described the creation of transgenic mouse lines expressing various levels of hPF4 RNA (26). We now measured total platelet hPF4 compared to the average hPF4 content of 4 human platelet controls. hPF4 levels varied from ˜0.5 times the content of human platelets in hPF4 Low mice (which have 1 copy of the hPF4 transgene/haploid genome) to ˜2 times the level in hPF4 Mid mice (which have 6 copies/haploid genome) to 6 times in hPF4 High mice (which have 22 copies/haploid genome) (FIG. 4A). Flow cytometric studies of platelets from these transgenic lines demonstrate that all have detectable surface-bound hPF4 in vivo measured 10 mins after IV injection of KKO, with antibody binding proportional to platelet PF4 expression (FIG. 4B).

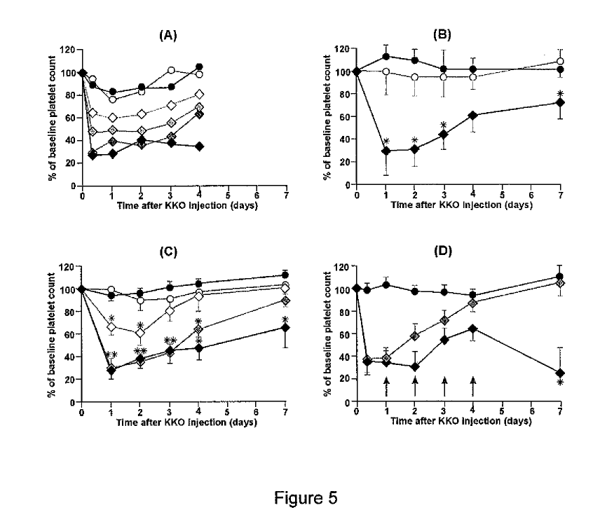

By 3 hr after an IP injection of KKO, hPF4^{High}/FcγRIIA⁺ mice developed severe, antibody dose-dependent thrombocytopenia, which persisted for >7 days (FIG. 5A). Mice that were hPF4^{High} or FcγRIIA alone did not develop thrombocytopenia (FIG. 5A). The severity of thrombocytopenia correlated with the dose of injected antibody. Thrombocytopenia was not seen with an equivalent amount of the isoimmune control TRA or RTO (FIG. 5B). The severity of the thrombocytopenia also correlated with the genetically determined level of hPF4 (FIG. 5C). Daily injections of 20 U of SQ heparin into hPF4^{High}/FcγRIIA⁺ mice as used in the previously described HIT murine model (14) did not lower the initial nadir platelet count further, but did prolong the duration of severe thrombocytopenia for >2 weeks (FIG. 5D and data not shown). There was no unexpected loss of animals in these studies, although specific histological studies for thrombotic events were not pursued.

Therapeutic Intervention in the Murine Model.

The above studies suggest that interventions that skew the GAG:PF4 ratio towards either extreme may protect against formation of HIT antigenic complexes on the platelet surface. We employed two such strategies to test this hypothesis: 1) Based on the data in FIG. 1B, we inferred that a marked excess of heparin would reduce surface antigenicity and prevent HIT even in the presence of a pathogenic anti-PF4/heparin antibody. 2) The data suggest a similar outcome would be expected from an excess of a cationic moiety that binds to platelets and prevents incorporation of PF4 into the antigenic complexes. Protamine sulfate is a small positively-charged molecule that competes with PF4 for binding to GAGs (33) and has been used clinically to neutralize heparin (34). Although cardiovascular side-effects have been reported rarely (35,36), it has the advantage over infusing large amounts of hPF4 as this approach should not give rise to a transient increase in surface antigenicity.

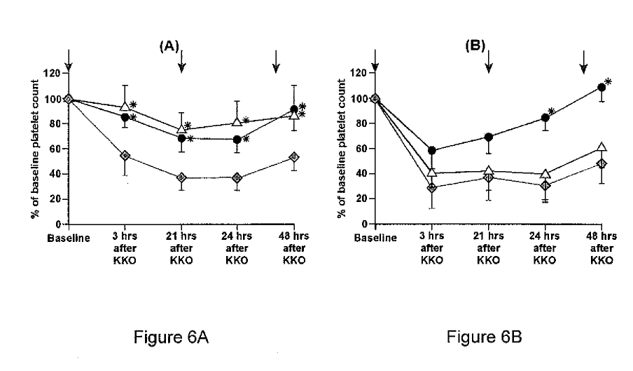

Transgenic hPF4^{Mid}/FcγRIIA⁺ mice were given an IV infusion of either 100 U/kg of unfractionated heparin or 2 mg/kg protamine sulfate 1 hr prior to an IP injection of 200 μg of KKO. Both prevented thrombocytopenia at 3 hr and decreased the severity of thrombocytopenia at 24 hr (FIG. 6A). Repeat doses given on the second and third days maintained platelet counts above the level in mice that had received KKO alone. In hPF4^{High}/FcγRIIA⁺ mice, these treatment regimens gave different results (FIG. 6B): High-dose heparin was ineffective in preventing KKO-induced thrombocytopenia. In contrast, platelet counts were significantly higher in protamine sulfate-treated mice than in mice receiving antibody alone at 24 and 48 hr.

### DISCUSSION

Surface-bound PF4 is antigenic for HIT antibodies and KKO over a narrow range of PF4 concentrations, leading to platelet activation through FcγRIIA. Our data suggest that PF4 forms antigenic complexes with endogenous GAGs on the surface of platelets similar to ULCs that form between HMW heparin and PF4 in solution (23). These data could explain why only a subgroup of heparinized patients with HIT antibodies develop HIT. Platelets from different individuals in the population show wide variation in PF4 content (unpublished data) and perhaps in released PF4 and surface PF4 levels. Those individuals with the highest levels of surface PF4 prior to heparinization appear to continue to express surface HIT antigenic complexes after heparinization and develop HIT. In addition, the proposed model may also help explain why HIT can develop after heparin therapy has been stopped (37) and why HIT can occur in a delayed fashion long after infused heparin has been cleared (38).

PF4 is a member of the CXC subfamily of chemokines that possesses high affinity for heparin and other large, anionic molecules (39). PF4 is expressed in megakaryocytes and stored in platelet α-granules from which it is released upon activation (40,41). After its release, PF4 binds to GAG on vascular cell surfaces (42). HIT IgG and the mAb KKO bind to Chinese Hamster Ovarian (CHO) cells in the presence of exogenous PF4 , but not to CHO cells lacking heparan sulfate- or chondroitin sulfate-containing proteoglycans (27). Similarly, these antibodies bind directly to monocytes (43) and cultured endothelial cells (44), and binding is reduced by pretreating with heparanases (44). Under certain experimental circumstances, heparin has been shown to promote the binding of HIT IgG and KKO to activated platelets, which acts in a feed-forward manner to perpetuate platelet activation and more IgG binding (22,45).

The concentration of PF4 that optimized KKO platelet binding (50 μg/mL) is the same as proved optimal for activation by HIT IgG (FIGS. 1A and 3A, respectively), and is well within what is attained in the immediate environ of activated platelets after platelet a-granular release (unpublished data). Moreover, the heparin concentrations (6.3-25 μg/mL, FIG. 1B) that enhanced KKO binding to platelets at 200 μg/mL of PF4 fall within the therapeutic range of heparinization (0.2-0.7 U/mL) (46), so that the conditions we analyzed are achievable in vivo.

To study the in vivo relevance of our observation, we used the previously described murine HIT model (14). We, as others, had assumed that heparin would be a necessary component for thrombocytopenia to develop in this model. Contrary to expectations, heparin is not required to induce thrombocytopenia. The pathogenic relevance of surface PF4 expression was supported by in vivo studies in transgenic mice expressing varying amounts of PF4 in which the severity of KKO-induced thrombocytopenia induced was proportionate to total platelet (and surface) hPF4 content (FIG. 5C). The reason for the presence of hPF4 on the surface of these platelets is unclear. Unlike patients with HIT, mice have little vascular disease that would sustain platelet activation leading to PF4 release and surface-bound PF4 . Transgenic expression of hPF4 in the presence of the fiull complement of murine PF4 may have exceeded the storage content of serglycins (47) inside the α-granules of their platelets, resulting in the observed “leak” of hPF4 and allowing the murine platelets to simulate patients with ongoing activation and partial degranulation of their platelets that may predispose to HIT.

Based on our findings, we reasoned that we could interfere with the development of thrombocytopenia in the double-transgenic mice by altering the surface GAG:PF4 ratio on the platelets. In support of this concept, infusing either high doses of heparin or protamine sulfate prevented KKO-induced thrombocytopenia in hPF4^{Mid}/FcγRIIA⁺ mice (FIG. 6). In the hPF4^{High}/FcγRIlA mice, the same heparin dose was ineffective, in contrast to the protamine sulfate, which retained its efficacy (FIG. 6). The dose of heparin we used is often exceeded in clinical settings (48), and we have used higher doses of both agents safely in mice (29). However, these interventions were intended to test our model and the role of surface platelet PF4 in HIT antigenicity. Moreover, we have yet to determine whether similar strategies can reverse established thrombocytopenia or thrombosis. Clinically, direct thrombin inhibitors block the explosive amplification of thrombin on platelet activation and coagulation, but have not eliminated the occurrence of amputations and death in affected patients. It is possible that antibody-mediated platelet activation promotes thrombosis through additional mechanisms involving platelet adhesion to the vasculature (49) and platelet-leukocyte aggregation (50) that would be better addressed by an intervention that acts proximal to thrombin generation. Thus, we envisage similar strategies to those in FIG. 6 that could target these proximal HIT mechanisms and be used in combination with direct thrombin inhibitors.

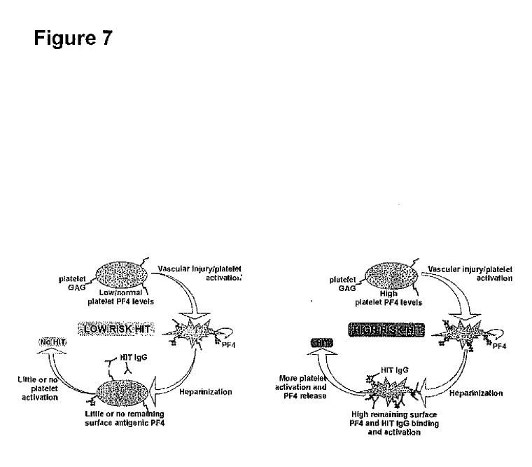

In FIG. 7, we propose a model for the onset of HIT based on our findings and on published literature. Patients who develop HIT are typically older and likely to have underlying cardiovascular disease and/or have undergone surgical manipulation. We propose that platelet activation in these patients leads to PF4 release and rebinding. In the vast majority of individuals and clinical settings, endogenous PF4 is low and surface PF4 expression does not exceed the equivalent of adding 50 μg/mL of PF4 (FIG. 7, left).

Therapeutic heparinization markedly reduces platelet surface PF4 . Heparinization would induce HIT antibody formation in up to half of these patients, but there would be little surface HIT antigen available and little risk of developing HIT. On the other hand, in the small number of individuals with high platelet PF4 content and sufficient platelet activation leading to high surface PF4 levels, therapeutic heparinization would not eliminate surface antigenicity (FIG. 7, right). These patients are at least as likely as other patients to develop HIT antibodies after heparinization. However in these individuals, the antibodies can activate a large number of platelets because of the high level of remaining platelet surface antigen, leading to more PF4 release and repetitive cycles of platelet activation. These patients are at high risk to develop HIT.

Our studies and model focus on the events on the platelet surface, but there is little reason to suppose that similar events are not concurrently happening on the surface of the endothelial lining, circulating monocytes and other vascular cells. The binding of HIT antibodies to the PF4 antigenic complexes on these cells would not only contribute to the developing thrombocytopenia, but also to the inflammatory state and to the thrombosis by expressing tissue factor and releasing procoagulant microparticles accelerating thrombin formation, that are recognized components of HIT (51-53). Finally, we believe that surface PF4 may have a biological role as well. We have previously shown that platelet PF4 content affects thrombogenicity in a bell-shaped curve fashion (29). We propose that both thrombogenicity and HIT antigenicity are greatest when formation of stable, GAG:PF4 antigenic complexes on cell surfaces is maximal. Based on the data presented herein, it appears that patients whose platelets retain surface antigenic complexes after heparinization are not only targets for HIT antibodies, but are also intrinsically prothrombotic.

In summary, the formation of HIT antigen on platelets occurs at specific concentrations of reactants. This can be demonstrated for binding of the mAb KKO to platelets and for FcγRIIA activation of platelets by KKO and by HIT IgG. When surface-bound PF4 exceeds this level, heparinization increases antigen formation. Murine models support the role of platelet surface PF4 complexes in the development of thrombocytopenia, and show that severity of thrombocytopenia depends on the level of platelet hPF4 . Infusions of either high doses of heparin or administration of protamine sulfate prevent the development of the thrombocytopenia in most settings, but heparin may be ineffective when the concentration of platelet PF4 is high. These data suggest that patients with high total and surface platelet PF4 expression may be at the highest risk to develop HIT when exposed to heparin and strategies to identify such patients and avoid heparin are warranted. Novel strategies to interfere with the formation of surface GAG:PF4 complexes suggested by this model may prove useful in the prevention and treatment of HIT.

### REFERENCES

1. Amiral J, Bridey F, Dreyfus M, Vissoc A M, Fressinaud E, Wolf M, Meyer D. Platelet factor 4 complexed to heparin is the target for antibodies generated in heparin-induced thrombocytopenia. Thromb Haemost 1992; 68:95-96.
2. Greinacher A, Potzsch B, Amiral J, Dummel V, Eichner A, Mueller-Eckhardt C. Heparin-associated thrombocytopenia: isolation of the antibody and characterization of a multimolecular PF4 -heparin complex as the major antigen. Thromb Haemost 1994; 71:247-251.
3. Arepally G, Reynolds C, Tomaski A, Amiral J, Jawad A, Poncz M, Cines D B. Comparison of PF4 /heparin ELISA assay with the 14C-serotonin release assay in the diagnosis of heparin-induced thrombocytopenia. Am J Clin Pathol 1995; 104:648-654.
4. Rhodes G R, Dixon R H, Silver D. Heparin induced thrombocytopenia: eight cases with thrombotic-hemorrhagic complications. Ann Surg 1977; 186:752-758.
5. Lee D H, Warkentin T E. Frequency of heparin-induced thrombocytopenia. In: Heparin-Induced Thrombocytopenia. Warkentin T, Greinacher A, eds. Marcel-Decker, Inc. New York, N.Y. p 107-148, 2000.
6. Walenga J M, Jeske W P, Messmore H L. Mechanisms of venous and arterial thrombosis in heparin-induced thrombocytopenia. J Thromb Thrombolysis 2000; 10:13-20.
7. Matthai W H Jr. Thrombocytopenia in cardiovascular patients: diagnosis and management. Chest 2005; 127:46S-52S.
8. Warkentin T E, Greinacher A. Heparin-induced thrombocytopenia: recognition, treatment, and prevention: the Seventh ACCP Conference on Antitlirombotic and Thrombolytic Therapy. Chest 2004; 126:311S-337S.
9.Lewis B E, Wallis D E, Leya F, Hursting M J, Kelton J G; Argatroban-915 Investigators. Argatroban anticoagulation in patients with heparin-induced thrombocytopenia. Arch Intern Med 2003; 163:1849-1856.
10. Lubenow N, Eichler P, Lietz T, Farner B, Greinacher A. Lepirudin for prophylaxis of thrombosis in patients with acute isolated heparin-induced thrombocytopenia: an analysis of 3 prospective studies. Blood 2004; 104:3072-3077.
11. Nand S, Wong W, Yuen B, Yetter A, Schmulbach E, Gross Fisher S. Heparin-induced thrombocytopenia with thrombosis: incidence, analysis of risk factors, and clinical outcomes in 108 consecutive patients treated at a single institution. Am J Hematol 1997; 56:12-16.
12. Kelton J G, Sheridan D, Santos A, Smith J, Steeves K, Smith C, Brown C, Murphy W G. Heparin-induced thrombocytopenia: laboratory studies. Blood 1988; 72:925-930.
13. Chong B H, Castaldi P A, Berndt M C. Heparin-induced thrombocytopenia: effects of rabbit IgG, and its Fab and FC fragments on antibody-heparin-platelet interaction. Thromb Res 1989; 55:291-295.
14. Reilly M P, Taylor S M, Hartman N K, Arepally G M, Sachais B S, Cines D B, Poncz M, McKenzie S E. Heparin-induced thrombocytopenia/thrombosis in a transgenic mouse model requires human platelet factor 4 and platelet activation through FcgammaRIIA. Blood 2001; 98:2442-447.
15. Bauer T L, Arepally G, Konkle B A, Mestichelli B, Shapiro S S, Cines D B, Poncz M, McNulty S, Amiral J, Hauck WW, Edie R N, Mannion J D. Prevalence of heparin-associated antibodies without thrombosis in patients undergoing cardiopulmonary bypass surgery. Circulation 1997; 95:1242-1246.
16. Visentin G P, Malik M, Cyganiak K A, Aster R H. Patients treated with unfractionated heparin during open heart surgery are at high risk to form antibodies reactive with heparin:platelet factor 4 complexes. J Lab Clin Med 1996; 128:376-383.
17. Pouplard C, May M A, Jochmann S, Amiral J, Vissac A M, Marchand M, Gruel Y. Antibodies to platelet factor 4-heparin after cardiopulmonary bypass in patients anticoagulated with unfractionated heparin or a low-molecular-weight heparin: clinical implications for heparin-induced thrombocytopenia. Circulation 1999; 99:2530-2536.
18. Suh J S, Malik M I, Aster R H, Visentin G P. Characterization of the humoral immune response in heparin-induced thrombocytopenia. Am J Hematol. 1997; 54:196-201.
19. Amiral J, Pouplard C, Vissac A M, Walenga J M, Jeske W, Gruel Y. Affinity purification of heparin-dependent antibodies to platelet factor 4 developed in heparin-induced thrombocytopenia: biological characteristics and effects on platelet activation. Br J Haematol 2000; 109:336-341.
20. Untch B, Ahmad S, Jeske W P, Messmore H L, Hoppensteadt D A, Walenga J M, Lietz H, Fareed J. Prevalence, isotype, and functionality of antiheparinplatelet factor 4 antibodies in patients treated with heparin and clinically suspected for heparin-induced thrombocytopenia. The pathogenic role of IgG. Thromb Res 2002; 105:117-123.
21. Refaai M A, Laposata M, Van Cott E M. Clinical significance of a borderline titer in a negative ELISA test for heparin-induced thrombocytopenia. Am J Clin Pathol 2003; 119:61-65
22. Home M K 3rd, Hutchison K J. Simultaneous binding of heparin and platelet factor-4 to platelets: further insights into the mechanism of heparin-induced thrombocytopenia. Am J Hematol 1998; 58:24-30.
23. Rauova L, Poncz M, McKenzie S E, Reilly M P, Arepally G, Weisel J W, Nagaswami C, Cines D B, Sachais B S. Ultralarge complexes of PF4 and heparin are central to the pathogenesis of heparin-induced thrombocytopenia. Blood 2005; 105:131-138.
24. Visentin G P, Moghaddam M, Beery S E, McFarland J G, Aster R H. Heparin is not required for detection of antibodies associated with heparin-induced thrombocytopenia/thrombosis. J Lab Clin Med 2001; 138:22-31.
25. Park K S, Rifat S, Eck H, Adachi K, Surrey S, Poncz M. Biologic and biochemic properties of recombinant platelet factor 4 demonstrate identity with the native protein. Blood 1990; 75:1290-1295.
26. Zhang C, Thornton M A, Kowalska M A, Sachis B S, Feldman M, Poncz M, McKenzie S E, Reilly M P. Localization of distal regulatory domains in the megakaryocyte-specific platelet basic protein/platelet factor 4 gene locus. Blood 2001; 98:610-617.
27. Arepally G M, Kamei S, Park K S, Kamei K, Li Z Q, Liu W, Siegel D L, Kisiel W, Cines D B, Poncz M. Characterization of a murine monoclonal antibody that mimics heparin-induced thrombocytopenia antibodies. Blood 2000; 95: 1533-1540.
28. Warkentin T E. Clinical presentation of heparin-induced thrombocytopenia. Semin Hematol 1998; 35:9-16.
29. Eslin D E, Zhang C, Samuels K J, Rauova L, Zhai L, Niewiarowski S, Cines D B, Poncz M, Kowalska M A. Transgenic mice studies demonstrate a role for Platelet Factor 4 in thrombosis: Dissociation between anticoagulant and antithrombotic effect of heparin. Blood 2004; 104:3173-3180.
30. McKenzie S E, Taylor S M, Malladi P, Yuhan H, Cassel D L, Chien P, Schwartz E, Schreiber A D, Surrey S, Reilly M P. The role of the human Fc receptor Fc gamma RIIA in the immune clearance of platelets: a transgenic mouse model. J Immunol 1999; 162:4311-4318.
31. Nader H B. Characterization of a heparan sulfate and a peculiar chondroitin 4-sulfate proteoglycan from platelets. Inhibition of the aggregation process by platelet chondroitin sulfate proteoglycan. J Biol Chem 1991; 266:10518-10523.
32. Handin R I, Cohen H J. Purification and binding properties of human platelet factor four. J Biol Chem 1976; 251 :4273-4282.
33. Shanberge J N, Quattrociocchi-Longe T M, Martens M H. Interrelationship of protamine sulfate and platelet factor 4 in the neutralization of heparin. Thromb Res 1987; 46:89-100.
34. Hawksley M. De-heparinisation of blood after cardiopulmonary bypass. Lancet 1966; 1:563-565.
35. Panos A, Orrit X, Chevalley C, Kalangos A. post-cardiotomy outcome, due to severe anaphylactic reaction to protamine. Eur J Cardiothorac Surg 2003; 24:325-327.
36. Viaro F, Dalio M B, Evora P R. Catastrophic cardiovascular adverse reactions to protamine are nitric oxide/cyclic guanosine monophosphate dependent and endothelium mediated: should methylene blue be the treatment of choice? Chest 2002; 122:1061-106.
37. Lewis B E, Walenga J M, Wallis D E. Anticoagulation with Novastan (argatroban) in patients with heparin-induced thrombocytopenia and heparininduced thrombocytopenia and thrombosis syndrome. Semin Thromb Hemost 1997; 23:197-202.
38. Warkentin T E, Kelton J G. Delayed-onset heparin-induced thrombocytopenia and thrombosis. Ann Intern Med 2001; 135:502-506.
39. Greinacher A, Alban S, Dummel V, Franz G, Mueller-Eckhardt C. Characterization of the structural requirements for a carbohydrate based anticoagulant with a reduced risk of inducing the immunological type of heparinassociated thrombocytopenia. Thromb Haemost 1995; 74:886-892.
40. Lane D A, Ireland H, Wolff S, Ranasinghe E, Dawes J. Detection of enhanced in vivo platelet alpha-granule release in different patient groups—comparison of beta-thromboglobulin, platelet factor 4 and thrombospondin assays. Thromb Haemost 1984; 52:183-187.
41. Holt J C, Niewiarowski S. Biochemistry of alpha granule proteins. Semin Hematol 1985; 22:151-163.
42. Rao A K, Niewiarowski S, James P, Holt J C, Harris M, Elfenbein B, Bastl C. Effect of heparin on the in vivo release and clearance of human platelet factor 4. Blood 1983; 61:1208-1214.
43. Arepally G M, Mayer I M. Antibodies from patients with heparin-induced thrombocytopenia stimulate monocytic cells to express tissue factor and secrete interleukin-8. Blood 2001; 98:1252-1254.
44. Cines D B, Tomaski A, Tannenbaum S. Immune endothelial-cell injury in heparin-associated thrombocytopenia. N Engl J Med 1987; 316:581-589.
45. Newman P M, Chong B H. Heparin-induced thrombocytopenia: new evidence for the dynamic binding of purified anti-PF4 -heparin antibodies to platelets and the resultant platelet activation. Blood 2000; 96:182-187.
46. Zanke B, Shojania A M. Comparison of two APTT methods of monitoring heparin therapy. APTT ratio and heparin response of pooled normal plasma. Am J Clin Pathol 1990; 93:684-689.
47. Kolset S O, Mann D M, Uhlin-Hansen L, Winberg J O, Ruoslahti E. Serglycin binding proteins in activated macrophages and platelets. J Leukoc Biol 1996; 59:545-554.
48. Murphy J A, Savage C M, Alpard S K, Deyo D J, Jayroe J B, Zwischenberger J B. Low-dose versus high-dose heparinization during arteriovenous carbon dioxide removal. Perfusion 2001; 16:460-468.
49. Blank M, Shoenfeld Y, Tavor S, Praprotnik S, Boffa M C, Weksler B, Walenga M J, Amiral J, Eldor A. Anti-platelet factor 4/heparin antibodies from patients with heparin-induced thrombocytopenia provoke direct activation of microvascular endothelial cells. Int Immunol 2002; 14:121-129.
50. Khairy M, Lasne D, Amelot A, Crespin M, Rendu F, Aiach M, Bachelot-Loza C. Polymorphonuclear leukocyte and monocyte activation induced by plasma from patients with heparin-induced thrombocytopenia in whole blood. Thromb Haemost 2004; 92:1411-1419.
51. Arepally G M, Mayer I M. Antibodies from patients with heparin-induced thrombocytopenia stimulate monocytic cells to express tissue factor and secrete interleukin-8. Blood 2001; 98:1252-1254.
52. Pouplard C, Iochmann S, Renard B, Herault O, Colombat P, Amiral J, Gruel Y. Induction of monocyte tissue factor expression by antibodies to heparin platelet factor 4 complexes developed in heparin-induced thrombocytopenia. Blood 2001; 97:3300-3302.
53. Warner M N, Pavord S, Moore J C, Warkentin T E, Hayward C P, Kelton J G. Serum-induced platelet procoagulant activity: an assay for the characterization of prothrombotic disorders. J Lab Clin Med 1999; 133:129-133.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for diagnosing a patient's predisposition for the development of heparin-induced thrombocytopenia (HIT), comprising;
a) obtaining a cell-containing biological sample from said patient; and
b) determining the level of at least one of
i) total platelet factor 4 (PF4 ) expression;
ii) surface PF-4 expression;
ii) expression of antigenic complexes comprising of PF4 and glycosaminoglycans on the surface of said cells,
elevated levels of said PF4 or said antigenic complexes being indicative of a predisposition for the development of HIT.

2. The method of claim 1, wherein said antigenic complexes are reactive with KKO antibody.

3. The method of claim 1, wherein said antigenic complexes are reactive with HIT immunoglobulin.

4. The method of claim 1, wherein said biological samples comprises cells selected from the group consisting of platelets, endothelial cells and monocytes.

5. The method of claim 4, wherein said sample comprises platelets.

6. The method of claim 5, optionally comprising treating the sample of step a) with PGE₁ to inhibit spontaneous activation of said platelets.

7. The method of claim 1, optionally comprising determining IgG titers in said patient.

8. The method of claim 1, wherein said cells are platelets and said glycosaminoglycan comprises chondroitin sulfate.

9. The method of claim 6, wherein antigenic levels of PF4 are determined using KKO antibody.

10. The method of claim 1, wherein said PF4 expression level is determined using a method selected from the group consisting of ELISA, radioimmunoassay, flow cytometry, histocytochemistry, and SDS-PAGE.

11. A method for identifying agents useful for the treatment of heparin induced thrombocytopenia comprising;
a) providing cells which express GAG-PF4 antigenic complexes on the surface;
b) incubating said cells in the presence and absence of said agent; and
c) identifying those agents which disrupt GAG-PF4 antigenic complexes, said agents having efficacy for the treatment of heparin induced thrombocytopenia, with the proviso that said agent is not heparin or protamine sulfate.

12. The method of claim 11, wherein said cells are selected from the group consisting of platelets, endothelial cells and monocytes.

13. The method of claim 11, wherein said cells are platelets.

14. The method of claim 11, wherein said complexes are detected with KKO antibody.

15. The method of claim 11, wherein said complexes are reactive with HIT immunoglobulin.
